(19) United States
(12) Patent Application Publication
(10) Pub. No.: US 2023/0314396 A1
(43) Pub. Date: Oct. 5, 2023

(54) SYSTEM AND METHOD FOR MICRODIALYSIS FOR IN-VITRO RELEASE TESTING OF DOSAGE FORMS

(71) Applicant: ORTIV-Q3 RESEARCH PVT. LTD, Ahmedabad (IN)

(72) Inventors: Mukesh KUMAR, Ahmedabad (IN); Pawan Kumar GUPTA, Ahmedabad (IN); Ashish Kumar GUPTA, Ahmedabad (IN)

(21) Appl. No.: 17/919,622

(22) PCT Filed: Apr. 20, 2021
(86) PCT No.: PCT/IN2021/050388

§ 371 Date: Oct. 18, 2022

(30) Foreign Application Priority Data
Apr. 20, 2020 (IN) 201921042553

(51) Int. Cl. G01N 33/15; B01L 3/00; B01L 7/00; G01N 21/33

(52) U.S. Cl. CPC G01N 33/15; B01L 3/502746; B01L 7/00; G01N 21/33; B01L 2300/18; B01L 2300/0654; B01L 2400/0487; B01L 2200/16; B01L 2300/0681

(57) ABSTRACT

The present invention relates to an improved microdialysis method utilizing an ultrafiltration module comprising of a plurality of hollow fiber microtubular membranes for In-Vitro Release Testing (IVRT) of dispersed dosage forms, such as but not limited to solutions, emulsions, suspensions, liposomes, nanodispersions, nanocrystals and polymeric nanocarriers. The invention further relates to a microdialysis based method providing an increase permeability area for faster release of an agent, which enables the substantial or near complete drug release in a shorter duration of time. The invention further relates to a system for in-vitro release testing of a dosage form with increased permeability area for faster release of an agent which enables the near complete or substantial release of a drug.

This patent application is the National Stage of International Application No. PCT/IN2021/050388 filed Apr. 20, 2021, which claims the benefit of priority from IN201921042553 filed Apr. 20, 2020, teachings of each of which are incorporated herein by reference in their entirety.

### DESCRIPTION

### Field of the Invention

The invention relates to an improved microdialysis method utilizing an ultrafiltration module comprising of a plurality of hollow fiber microtubular membranes for In-Vitro Release Testing (IVRT) of dispersed dosage forms, such as but not limited to solutions, emulsions, suspensions, liposomes, nanodispersions, nanocrystals and polymeric nanocarriers. The invention further relates to a microdialysis based method providing an increase permeability area for faster release of an agent, which enables the substantial or near complete drug release in a shorter duration of time. The invention further relates to a system for in-vitro release testing of a dosage form with increased permeability area for faster release of an agent which enables the near complete or substantial release of a drug.

### Background of the Invention

Nanoparticulate dispersed systems have emerged as prevalent drug delivery systems over the past few decades. These delivery systems, such as liposomes, emulsions, suspensions nanocrystals, and polymeric nanocarriers have been extensively used to improve bioavailability, prolong pharmacological effects, achieve targeted drug delivery, as well as reduce side effects. Considering that any unanticipated change in product performance of such systems may result in toxicity and/or change in-vivo efficacy, it is essential to develop suitable in-vitro release testing methods to ensure product quality and performance, and to assist in product development. Such methods should be able to capture differences or changes in Critical Quality Attributes (CQA) and Critical Process Parameters (CPP).

In-vitro release testing methods also play an important role in bioequivalence determination. Regulatory authorities worldwide are considering in-vitro release testing as part of totality of evidence approach to demonstrate product sameness to the innovator. Once validated, in-vitro release testing can also be used as a specification. It also provides a potential tool for acceptability of post-approval manufacturing changes.

In order to ensure product performance and assist in product development, extensive efforts have been made to develop suitable in-vitro release testing methods for nanoparticulate delivery systems.

These methods can be broadly divided into two categories:

The Standardized methods are as following:
Paddle method (USP Apparatus II, Apparatus II with enhancer cell)
Flow-through system (USP-type IV)

The Non-Standardized methods are as following:
Dialysis sac
Reverse dialysis sac
Controlled temperature Shaking bath (sample and separate).

Despite the availability of several in-vitro release testing methodologies, there are challenges associated with in-vitro release testing method development for complex formulations especially ophthalmic drug products. The regulatory authorities have several expectations in view of the potential role of in-vitro release testing in establishing bioequivalence. These expectations are highlighted below:
Ophthalmic dosage form has a complex route of delivery as the drug is distributed in several phases and there are a complex set of conditions governing release. Particularly, the residence time is quite short in the ocular region; the similar time frame is expected in in-vitro release testing methodology.
The in-vitro release testing method should detect meaningful variations in formulation and manufacturing parameters.
An ideal test accounts for factors physiologically relevant to the in-vivo conditions
   Should avoid the excessive dilution.
   Should avoid the use of surfactant, organic solvents (or justify, if required).
Optimization/Justification required for media, membrane, and temperature selection.
Information on drug adsorption, diffusion resistance and adequacy of mol. wt. cut off.
Drug release should be complete (>75%, reach a plateau) or provide justification.
Ideally, the in-vitro release testing method should be able to discriminate batches that are not bioequivalent.

Microdialysis is currently being explored as one of the alternative techniques for IVRT evaluation of dispersed dosage forms. In the past it has been extensively used as one of the in vivo release evaluation tool for continuous measurement of free, unbound analyte concentrations in the extracellular fluid of virtually any tissue. Analytes may include endogenous molecules, e.g. neurotransmitter, hormones, glucose, etc. to assess their biochemical functions in the body, or exogenous compounds—e.g. pharmaceuticals- to determine their distribution within the body.

Microdialysis has contributed with very important knowledge to the understanding of target-specific concentrations and their relationship to pharmacodynamic effects from a systems pharmacology perspective, aiding in the global understanding of drug effects. Therefore, an important role of microdialysis in systems pharmacology is to measure and identify target site concentrations that may differ from plasma concentrations due to distributional consequences of transporters and other processes and in relating these concentrations to pharmacodynamic measurements.

The microdialysis probe is designed to mimic a blood capillary and consists of a shaft with a semipermeable hollow fiber membrane at its tip, which is connected to inlet and outlet tubing. The probe is continuously perfused with an aqueous solution (perfsate) that closely resembles the (ionic) composition of the surrounding tissue fluid at a low flow rate of approximately 0.1-5 μL/min. Once inserted into the tissue or (body) fluid of interest, small solutes can cross the semi permeable membrane by passive diffusion. The direction of the analyte flow is determined by the respective concentration gradient and allows the usage of microdialysis probes as a sampling tool. The solution leaving the probe (dialysate) is collected at certain time intervals for analysis.

Microdialysis methods have also been applied to limited extent in IVRT studies of dispersed dosage forms. Microdialysis can offer significant advantages compared to other IVRT methods. For instance, since microdialysis probes are very small, they can be placed directly into small “receivers” for in-vitro systems. Also, the method offers the advantage of a clean aqueous sample without pre-detection sample preparation, such as separation or clean up steps. It also enables quick and frequent sampling during a shorter duration of time.

The prior art technique is based on the dialysis principle, employing a “semipermeable” membrane, i.e., one that is highly permeable to water and small molecules. In this method, a sampling solution is perfused continuously through a micro capillary probe, and an agent such as a drug or other material of interest passively diffuses into the dialysate from the surrounding medium. The sampling solution is collected and analyzed for the drug content, and the concentration of drug or other material of interest in the surrounding medium is then estimated from that information. This technique is poorly suited for studies in which concentrations change relatively rapidly. The inability of continuous flow microdialysis to sample every 10-15 seconds is a disadvantage. As well as typical perfusion flow rates, the recovery of the drug and the resulting sampling efficiency can be poor. It is also known as reverse dialysis method wherein the dosage form can be passed through the probe and the drug diffused into outer dialysate is estimated quantitatively.

The other reported IVRT approach for microdialysis is the pulsatile microdialysis (PMD) technique. In this method, the sampling solution (dialysate) is pumped into the probe and then allowed to remain at rest for a brief, the discrete period referred to as the resting time. After a suitable resting time (typically 3-100 seconds, preferably 3-15 seconds), the dialysate is flushed (i.e. pumped) out and collected for assay. It is usually preferred that this flushing is done as a single pulse at a relatively high flow rate (typically 50-165 pUImin), preferably to minimize or eliminate the effects of further diffusion, which usually simplifies the mathematical analysis of the data.

Some of the in vivo and in vitro methods or techniques for determination of drug content or drug release that have been reported are as follows:
1) K. B. Shah et. al. in International Journal of Pharmaceutics, 48, 64-74, 2014 describe an improved method for the characterization of supersaturation and precipitation of poorly soluble drugs using pulsatile microdialysis (PMD).
2) Margareta Hammarlund-Udenaes in the AAPS Journal, J9(5), 1294-1303, September 2017 has done a very methodical review of microdialysis as an important technique in systems pharmacology
3) S. B. Jadhav et. al. in Journal of Pharmaceutical Sciences, 1-10, 2016 have provided an excellent treatise on microdialysis of large molecules.
4) K. B. Rittenhouse et. al. in Advanced Drug Delivery Reviews, § 5, 229-241, 2000 have dealt in details on drug delivery to the eye by microdialysis methods.
5) D. Solomon et. al. in the AAPS Journal, September 2017, have provided a detailed review of the role of in vitro release methods in liposomal formulation development from a regulatory perspective.
6) R. B. Bellantone in U.S. Pat. Nos. 8,333,107 B2; 8,647,295 B2 and 8,652,088B2 discloses an improved method of effecting and measuring mass transfer by microdialysis of relatively small quantities of dissolved, suspended or otherwise dispersed material between two media existing on the inner and outside of the microdialysis probe
7) D. Patel et. al. in Journal of Controlled Release, 333, 65-75, 2021 describe an in vitro release test (IVRT) of complex drug products by adaptive perfusion.
8) L. B. Belintong et. al. in Chinese Patent No. 2114020041U disclose a system for accurately determining apparent supersaturated solubility and rate allowing very accurate and rapid measurement of mass transfer by allowing the desired measurement reagent to diffuse into and out of a small, very accurate volume of microdialysis probe(s) and then using the known volume of liquid as a single pulse rapid pumping or flushing (pulsing) probe. The system is based on the mathematical operation of Fick's law. Experimental data validation of the resulting equation was performed using Azofamide, Warfarin and Benzocaine as the test drugs.
9) F. Feichtner et. al. in U.S. Pat. No. 8,535,537 B2 disclose a device, apparatus and a method for monitoring a parameter of a fluidic sample by microdialysis, the device comprising a permeable membrane that has a first surface to be brought in contact with the fluidic sample to traverse the permeable membrane and a second surface to be brought in contact with a dialysis fluid, wherein the first surface is smoother than the second surface.
10) S. H. S. B. Boddu et. al. in Bioanalysis, 2(3), 487-507, 2010 have dealt with in detail on ocular microdialysis, a continuous sampling technique to study the pharmacokinetics and pharmacodynamics in the eye.
11) R. Klaus et. al. in Journal of Ocular Pharmacology and Therapeutics, 1-6, 2016 have reported an exploratory microdialysis study to assess the ocular pharmacokinetics of Ciprofloxacin eye drops in rabbits.
12) C. S. Dias et. al. in Investigative Opthalmology & Visual Science, 44(1), 300-305, January 2013 have reported a method of microdialysis to study posterior segment ocular pharmacokinetics in conscious rabbit models.
13) B. S. Anand et. al. in International Journal of Pharmaceutics, 281, 79-88, 2004 have carried out a study to validate a novel ocular microdialysis sampling technique in rabbits with permanently implanted vitreous probes.
14) M. K. Hidau et. al. in a Poster No. 33T0200 in 2016 AAPS Conference held in the Colorado Convention Center, Denver from Nov. 13-17, 2016 have exhibited the development towards in vitro drug release methods for Difluprednate ophthalmic emulsion.

However, the available microdialysis IVRT methods are associated with certain deficiencies such as
Not achieving the substantial or near complete drug release as per the regulatory expectations drug release since the permeation window is very small and is usually indicated by drug flux across a specified area, whereas the drug release is expected to be substantial or near complete (>75%), and
The existing methods are not Quality Control friendly as the commercially available probe are quite delicate, sophisticated and require specialized pumping mechanism to handle flow volumes in micro liter range (e.g. syringe pump).
The existing methods and systems are quite expensive keeping requirement of multiple channels monitoring in view.
The setting up of such systems for IVRT requires complex setup procedures & are not very robust.
The existing systems are not amenable to compendial IVRT devices such as United States Pharmacopeia (USP) Type1/2 & Type 4.

Therefore, to overcome these certain deficiencies the inventors of the present invention have come up with an improved microdialysis method to provide complete and faster drug release in the in-vitro analysis of dosage forms and enable more frequent sampling in comparison to the known techniques.

### SUMMARY OF INVENTION

An aspect of the present invention provides a system for in-vitro release testing of a drug from the dosage form comprising:
a) a media reservoir configured to hold the dissolution media wherein the media reservoir consists of a sampling provision,
b) a first temperature controlling unit to maintain the temperature ofthe dissolution media wherein the media reservoir is kept in contact with the temperature controlling unit.
c) a pump connected to the media reservoir on one end and a hollow fiber module at the other end, wherein the pump is configured to pump the dissolution media from the media reservoir, and
d) a second temperature controlling unit may be kept in between the media reservoir and the plurality of hollow fiber module in the direction of the flow of the media to maintain the temperature of the recirculating media.
e) a hollow fiber module connected to the other end of the pump wherein the hollow fiber module comprises of:
   a housing,
   a permeate chamber for holding the sample,
   a retentate chamber comprising a plurality of hollow fiber microtubular membranes, with a total surface area of between 5 to 200 cm², preferably a total surface area of between 13 to 92 cm² wherein the drug is released from the dosage form into the retentate, wherein the retentate is recirculated back to the media reservoir.
      Optionally the retentate may be collected manually from the media reservoir.
      Optionally external fraction collector may be connected to the media reservoir for detecting the dispersed dosage form controlled by a firmware.

Another aspect of the present invention provides a system for in-vitro release testing of a drug from the dosage form which may include a first temperature controlling unit or a second temperature controlling unit or both the first temperature controlling unit and second controlling unit may be used in conjunction.

In yet another aspect, the present invention provides a system for in-vitro release testing of a drug from the dosage form comprising:
a) a media reservoir configured to hold the dissolution media wherein the media reservoir consists of a sampling provision,
b) a first temperature controlling unit to maintain the temperature of the dissolution media wherein the media reservoir is kept in contact with the temperature controlling unit.
   Optionally, a second temperature controlling unit may be kept in between the media reservoir and the hollow fiber module in the direction of the flow of the media to maintain the temperature of the dissolution media.
c) a pump connected to the media reservoir on one end and a hollow fiber module at the other end, wherein the pump is configured to pump the dissolution media from the media reservoir, and
d) a hollow fiber module connected to the other end of the pump wherein the hollow fiber module comprises of:
   a housing,
   a permeate chamber for holding the sample,
   a retentate chamber comprising a plurality of hollow fiber microtubular membranes, with a total surface area of between 5 to 200 cm², preferably a total surface area of between 13 to 92 cm², wherein the drug is released from the dosage form into the retentate, wherein the retentate is recirculated back to the media reservoir.
Optionally a UV spectrophotometric cell is connected in between the media reservoir and the hollow fiber module in the direction of the flow of the media for real time monitoring of the release agent.
Optionally positioning an optical fiber probe in the media reservoir for UV spectrophotometric real time detection of an agent in the dosage form.

In yet another aspect, the present invention provides a system for in-vitro release testing of a drug from the dosage form which may include a first temperature controlling unit or a second temperature controlling unit or both the first temperature controlling unit and second controlling unit may be used in conjunction.

In a particular aspect, the present invention relates to a microdialysis method for providing an increased permeability area for determination of the diffusible or free concentration of drug in a dosage form, the method comprising:
a) placing the dosage form in the permeate chamber of a hollow fiber module and closing the permeate chamber using end plugs.
b) pumping the dissolution medium from the media reservoir through the retentate chamber of hollow fiber module.
c) circulating the release media through retentate chamber comprising of the plurality of hollow fiber microtubular membranes, wherein release of the drug is occurring in the plurality of hollow fiber microtubular membranes, wherein the plurality of hollow fiber microtubular membrane has a total surface area of between 5 to 200 cm², preferably a total surface area of between 13 to 92 cm²,
d) maintaining the temperature of the dissolution media using the first temperature controlling unit (12) or a second temperature controlling unit (15) or a combination of both e) collecting the retentate from the release media through the sampling provision of media reservoir.
   Optionally the retentate is sampled from the external fraction collector connected to the media reservoir.
f) optionally detecting a component of the dispersed dosage form in the release medium controlled by a firmware.

In a particular aspect, the present invention relates to a microdialysis method for providing an increased permeability area for determination of the diffusible or free concentration of a drug in a dosage form, the method comprising:
a) placing the dosage form in the permeate chamber of a hollow fiber module.
b) pumping the dissolution medium from the media reservoir through the retentate chamber of hollow fiber module.
c) circulating the retentate media through the plurality of hollow fiber microtubular membranes of the retentate chamber resulting in the release media, wherein release of the drug is occurring in the plurality of hollow fiber microtubular membranes, wherein the plurality of hollow fiber microtubular membrane has a total surface area of between 5 to 200 cm², preferably a total surface area of between 13 to 92 cm².

Optionally circulating the release media through a UV spectrophotometric cell kept between the media reservoir and the hollow fiber module in the direction of the flow of the media for real time detection of an agent in the dosage form.
Optionally positioning an optical fiber probe in the media reservoir, wherein the optical fiber probe is connected to UV spectrophotometer for real time detection of an agent in the dosage form.
d) maintaining the temperature of the dissolution media using the first temperature controlling unit (12) or a second temperature controlling unit (15) or a combination of both.

In another aspect each hollow fiber module a provision for conditioning with appropriate washing fluid water or optionally water containing 10% Iso Propyl Alcohol (IPA) or optionally 0.5% surfactant such as but not limited to Sodium Lauryl Sulfate (SLS), Tween 80 to enable column reuse is provided.

In an embodiment (reverse process) the dosage form can be added to release media and the dispersed drug can be recirculated through the retentate chamber (microtubules) under set pressure. The permeate chamber shall remain open and the drug content in the permeate can be monitored by appropriate means.

In a preferred aspect, the present invention relates to a microdialysis method for the analysis of the ophthalmic dosage form for complete and faster drug release in a shorter duration of time.

In another aspect, the present invention relates to a microdialysis method for facilitation of physiological relevant in vitro release testing parameters for ophthalmic dosage forms i.e. quick and frequent sampling time and smaller release media volumes.

A further aspect of the present invention relates to a microdialysis method that provides an increased permeability area for faster release of the drug, enabling the near complete or substantial drug release in a shorter duration of time.

In another aspect of the present invention, the hollow fiber module comprises of a bundle of plurality of hollow fiber microtubules or microtubular membrane or microcapillaries.

In yet another aspect of the present invention, the microdialysis method for in-vitro release testing of the dosage form is selected from a group comprising of solutions, emulsion, suspension, liposome or nanodispersion, nanocrystals or polymeric nanocarriers.

In a yet another aspect of the present invention, the temperature controlling provision consists of one or two temperature control units comprising of high accuracy water bath, shaking water bath, magnetically stirred hotplates or any other heating device.

In a still further aspect of the present invention the location of first temperature controlling unit can be shifted from media reservoir to hollow fiber module to control the temperature of release media recirculating through it.

An aspect of the present invention provides a system for in-vitro release testing of a dosage form wherein the drug is released in the hollow fiber bundles of the retentate chamber in the hollow fiber module.

An aspect of the present invention provides a system for in-vitro release testing of a drug from dosage form for real time monitoring of pressure of the retentate or permeate fluid in the hollow fiber module.

An aspect of the present invention provides a system for in-vitro release testing of a drug from dosage form for real time monitoring of temperature of the release medium.

An aspect of the present invention provides a system for in-vitro release testing of a drug from dosage form for real time monitoring of flow rate of the release media

An aspect of the present invention provides a system for in-vitro release testing of a drug from dosage form for real time UV monitoring of the drug dispersed from the dosage form.

An aspect of the present invention provides a system for in-vitro release testing of a drug from dosage form for enabling offline sample collection of release media through an optional fraction collector.

An aspect of the present invention provides a system for in-vitro release testing of a drug from dosage form with the retentate media having a flow rate between 2-100 ml/min, more preferably 4-32 ml/min

An aspect of the present invention provides a system for in-vitro release testing of a drug from dosage form wherein the media volume that can be processed by the plurality of hollow fiber microtubular membranes is between 2 ml to 1000 ml, more preferably between 20 ml to 100 ml.

An aspect of the present invention provides a system for in-vitro release testing of a drug from dosage form with the plurality of hollow fiber microtubular membranes having a 3 KD to 1000 KD molecular weight cut off for dialysis.

An aspect the present invention provides a system for in-vitro release testing of a drug from dosage form wherein each hollow fiber module housing is made up of white polysulfone or polysulfone.

An aspect of the present invention provides a system for in-vitro release testing of a drug from dosage form with plurality of hollow fiber membrane having a surface area range of 2 to 200 cm².

An aspect of the present invention provides a hollow fiber module for in-vitro release testing of a drug from dosage form, comprising plurality of hollow fiber microtubular membrane, preferably more than two microtubules.

An aspect of the present invention provides a system for in-vitro release testing of a drug from dosage form wherein hollow fiber microtubule or microtubular membrane or microcapillaries comprises of semipermeable membranes, typically polysulfone, Polyethersulfon, mixed cellulose ester or Modified Polyethersulfone.

An aspect of the present invention provides a system for microdialysis based in-vitro release testing of a drug from dosage form optionally connected to modified USP type 1, 2 or USP type4 dissolution system.

Many other objects and advantages will be apparent from the following description of preferred embodiments when read in conjunction with the appended drawings.

### BRIEF DESCRIPTION OF THE DRAWING

FIG. 1: A system for in-vitro release testing of a drug from dosage form with automated and/or realtime monitoring of drug release.

FIG. 2: A system for in-vitro release testing of a drug from dosage form with offline monitoring of drug release comprising manual sampling.

FIG. 3: A graph showing results of IVRT of nepafenac ophthalmic suspension using USP Type-4 apparatus.

FIG. 4: A graph showing results of IVRT of cyclosporin ophthalmic emulsion using USP Type-4 apparatus.

FIG. 5: A graph showing results of IVRT cyclosporin ophthalmic emulsion using ready to use dialysis bag on USP Type-IV.

### DETAILED DESCRIPTION OF THE INVENTION

The terms disclosed herein are to be given their ordinary meaning in the art unless otherwise indicated. Other specifically defined terms are to be construed in a manner consistent with the definition provided herein. The definitions are provided to aid in describing particular embodiments and are not intended to limit the claimed disclosure. Unless otherwise defined, all technical and scientific terms used herein have the same meaning as commonly understood by one of ordinary person skilled in the art.

As used herein, the singular forms “a”, “an”, and “the” include plural referents unless the context clearly dictates otherwise.

As used herein, the terms “agent” and “drug” are often used interchangeably, and it will be further understood that the term “agent” is the broader term and is intended to include not only drugs but also any other pharmaceutically or chemically active material that will function in the present invention for any purpose for which the methods and mathematics of the present invention may be applied.

As used herein, the term “in-vitro” refers to an artificial environment and to processes or reactions that occur within an artificial environment. In-vitro environments can consist of but are not limited to, test tubes and cell culture.

As used herein, the term “dissolution apparatus” refers to a machine which may be used to determine dissolution characteristics (function) of a drug product such as solution, emulsion, suspension, liposome, nanodispersion, nanocrystals or polymeric nanocarriers.

As used herein, the term “hollow fiber microtubular membrane” refers to hollow fiber microtubule or microtubular membrane or microcapillaries comprising of semipermeable membranes, typically polysulfone, Polyethersulfon, cellulose esters or Modified Polyethersulfone.

Accordingly, the present invention relates a system for in-vitro release testing of an agent from the dosage form comprising:
a) a media reservoir (11) configured to hold the dissolution media wherein the media reservoir consists of a sampling provision,
b) a first temperature controlling unit (12) to maintain the temperature of the dissolution medium wherein the media reservoir is kept in contact with the temperature controlling unit.
Optionally, a second temperature controlling unit (15) may be kept in between the media reservoir and the hollow fiber module (17) in the direction of the flow of the media to maintain the temperature of the dissolution media. The first temperature controlling unit (12) or a second temperature controlling unit (15) may be used alone or both the first temperature controlling unit and second controlling unit may be used in conjunction.
c) a pump (14) connected to the media reservoir on one end and a hollow fiber module (17) at the other end, wherein the pump is configured to pump the dissolution media from the media reservoir, and
d) a hollow fiber module (17) connected to the other end of the pump wherein the hollow fiber module comprises of:
   a housing,
   a permeate chamber (171) for holding the sample,
   a retentate chamber (172) comprising a plurality of hollow fiber microtubular membranes, with a total surface area of between 5 to 200 cm², preferably a total surface area of between 13 to 92 cm² wherein the agent is released from the dosage form into the retentate, wherein the retentate is recirculated back to the media reservoir (11).
Optionally the retentate may be collected manually from the media reservoir (11).
Optionally external fraction collector (20) may be connected to the media reservoir (11) for detecting the dispersed dosage form controlled by a firmware.

The dosage form may be selected from solution, emulsion, suspension, liposome, nanodispersions, nanocrystals and polymeric nanocarriers. Suitable dosage forms include ophthalmic, topical, parenteral, liquid oral dosage form and the like. It is preferable to use ophthalmic dosage form in the present invention.

The temperature controlling unit (12 or 15) may be selected from shaking water bath, high accuracy water bath, hot plate, any other heating device and the like. There may be one or two temperature controlling units used in the present system for in vitro release testing of an agent from the dosage form. In one embodiment the temperature controlling unit (15) may be placed between the media reservoir (11) and the plurality of hollow fiber module in the direction of the flow of the media to maintain the temperature of the recirculating media. It is preferable to use high accuracy water bath.

The pump configured for circulating the media may be selected from peristaltic pump, syringe pump or piston pump.

The plurality of hollow fiber microtubules may include more than two microtubules. It is preferable to have at least five microtubules in the bundle. The hollow fiber module is an ultrafiltration module comprising of the bundle of hollow fiber microtubular (semipermeable) membranes. Such modules are commercially available from Repligen (MicroKros®) in different membranes like Modified Polyethersulfone (mPES), Polysulfone, Mixed cellulose esters, Polyethersulfones and molecular weights cut off (3 KD-750 KD). MicroKros®@modules have a total membrane surface area ranging from 13 to 92 cm² and can be used to process release media volume ranging from 4 ml to 1000 ml. There are bigger version MidiKros@ modules that have a total membrane surface area of 75 to 610 cm² to process release media volumes from 20 ml to 1000. The membranes with molecular weight cut off in the range of 0.05 micron to 0.5 micron and molecular weights cut off are also available as possible alternatives. Other modules include hollow fiber cartridges from Watersep@, which are offered in single use, autoclaveable and reusable formats which include the Green line@ single use hollow fiber cartridge, the Steamer line@ hollow fiber cartridges and the reuse line hollow fiber cartridges respectively. All 3 product lines are available with 3 different lengths, 12″-24″-41″ and in 8 cartridge sizes, with 13 membrane cut-offs and 3 fiber IDs. The Green Line and the Steamer Line incorporate a glycerin free, low extractable membrane that is ready to use and does not require any pre-flush. The ReUse Line can be cleaned, stored and used repeatedly. Fresenius medical care and Cytiva Lifesciences also offer hollow fiber modules but their devices are bigger in surface area and are typically more relevant for processing higher volumes in bioprocessing applications.

The housing of the hollow fiber module may be made of polysulfone, white polysulfone and the like.

The sample holder of the permeate chamber may include provision for holding various dosage forms. The dosage forms may be selected from ophthalmic, Parenteral, topical and oral dosage form and the like. It is more preferable for IVRT evaluation of ophthalmic and parenteral dispersed dosage forms.

The plurality of hollow fiber microtubular membrane may have a total surface area of between 5 to 200 cm². It is preferable to have a total surface area of 13 to 92 cm². The term hollow fiber microtubular membrane may encompass microtubular membrane or microcapillaries.

The drug may be released from the dosage form into the retentate.

The retentate may be recirculated back to the media reservoir (11).

In one embodiment the system the drug may be detected offline wherein the dispersed dosage form is detected by appropriate means via manual sample collection from the reservoir.

In one embodiment the location of first temperature controlling unit (12) can be shifted from media reservoir (11) to hollow fiber module (17) to control the temperature of release media recirculating through it.

Another embodiment of the present invention relates to a system for in-vitro release testing of an agent from the dosage form comprising:
a) a media reservoir (11) configured to hold the dissolution media wherein the media reservoir (11) consists of a sampling provision,
b) a first temperature controlling unit (12) to maintain the temperature of the dissolution medium wherein the media reservoir (11) is kept in contact with the temperature controlling unit.
   Optionally, a second temperature controlling unit (15) may be kept in between the media reservoir (11) and the hollow fiber module (17) in the direction of the flow of the media to maintain the temperature of the dissolution media. The first temperature controlling unit or a second temperature controlling unit may be used alone or both the first temperature controlling unit and second controlling unit may be used in conjunction.
c) a pump (14) connected to the media reservoir (11) on one end and a hollow fiber module at the other end, wherein the pump is configured to pump the dissolution media from the media reservoir (11), and
d) a hollow fiber module (17) connected to the other end of the pump wherein the hollow fiber module comprises of:
   a housing,
   a permeate chamber (171) for holding the sample,
   a retentate chamber (172) comprising a plurality of hollow fiber microtubular membranes, with a total surface area of between 5 to 200 cm², preferably a total surface area of between 13 to 92 cm², wherein the agent is released from the dosage form into the retentate, wherein the retentate is recirculated back to the media reservoir (11).
      Optionally a UV spectrophotometric cell (13) is connected in between the media reservoir (11) and the hollow fiber module (17) in the direction of the flow of the media for real time monitoring of the release agent.
      Optionally an optical fiber probe in the media reservoir (11) can be used for UV spectrophotometric real time detection of an agent in the dosage form.

The dosage form may be selected from solution, emulsion, suspension, liposome, nanodispersions, nanocrystals and polymeric nanocarriers. Suitable dosage forms include ophthalmic, parenteral, topical dosage form, and the like. It is preferable to use ophthalmic dosage form in the present invention.

The temperature controlling unit may be selected from shaking water bath, high accuracy water bath, hot plate, any other heating device and the like. There may be one or two temperature controlling units used in the present system for in vitro release testing of an agent from the dosage form. In one embodiment the temperature controlling unit may be placed between the media reservoir (11) and the plurality of hollow fiber module in the direction of the flow of the media to maintain the temperature of the recirculating media. It is preferable to use high accuracy water bath.

The pump configured for circulating the media may be selected from peristaltic pump, syringe pump or piston pump.

The plurality of hollow fiber microtubules may include more than two microtubules. It is preferable to have at least five microtubules.

The housing of the hollow fiber module may be made of polysulfone, white polysulfone and the like.

The sample holder of the permeate chamber may include provision for holding various dosage forms. The dosage forms may be selected from ophthalmic, topical dosage form, parenteral dosage form and the like. It is more preferable for evaluation of the ophthalmic dosage form.

The plurality of hollow fiber microtubular membrane may have a total surface area of between 5 to 200 cm². It is preferable to have a total surface area of 13 to 92 cm². The term hollow fiber microtubular membrane may encompass microtubular membrane or microcapillaries.

The plurality of hollow fiber microtubular membranes may have a 3 KD to 1000 KD molecular weight cut off for filtering. It is preferable to have a 50-750 KD Molecular weight cut off for dialysis.

The drug may be released from the dosage form into the retentate.

The retentate may be recirculated back to the media reservoir (11).

In one embodiment a real time monitoring of the release agent from the dosage form may be effected wherein the dispersed dosage form is detected by an online UV spectrophotometric cell (13). The spectrophotometric cell may be positioned between the media reservoir (11) and the hollow fiber module in the direction of the flow of the media.

In one embodiment the system for in-vitro release testing of a drug from the dosage form may be optionally used in conjunction with modified USP type 1/2 or 4 apparatus.

In another preferred embodiment, the present invention relates to a microdialysis method for providing an increased permeability area for determination of the diffusible or free concentration of drug in a dosage form, the method comprising:
a) placing the dosage form in the permeate chamber (171) of a hollow fiber module (17) and closing the permeate chamber using end plugs.
b) pumping the dissolution medium from the media reservoir (11) through the retentate chamber (172) of hollow fiber module (17).
c) circulating the release media through retentate chamber comprising of the plurality of hollow fiber microtubular membranes, wherein release of the drug is occurring in the plurality of hollow fiber microtubular membranes, wherein the plurality of hollow fiber microtubular membrane has a total surface area of between 5 to 200 cm², preferably a total surface area of between 13 to 92 cm².
d) maintaining the temperature of the dissolution media using the first temperature controlling unit (12) or a second temperature controlling unit (15) or a combination of both.
e) collecting the retentate from the release media through the sampling provision of media reservoir (11).
   Optionally the retentate is collected from the external fraction collector (20) connected to the media reservoir (11).
   Optionally detecting a component of the dispersed dosage form in the release medium controlled by a firmware (21).

In another embodiment of the present invention a microdialysis method for in-vitro release testing of a drug from dosage form for enabling offline sample collection of release media through an optional fraction collector (20) may be provided.

In yet another preferred embodiment, the present invention relates to a microdialysis method for providing an increased permeability area for determination of the diffusible or free concentration of drug in a dosage form, the method comprising:
a) placing the dosage form in the permeate chamber (171) of a hollow fiber module (17).
b) pumping the dissolution medium from the media reservoir (11) through the retentate chamber (172) of hollow fiber module.
c) circulating the retentate media through the plurality of hollow fiber microtubular membranes of the retentate chamber resulting in the release media, wherein release of the drug is occurring in the plurality of hollow fiber microtubular membranes, wherein the plurality of hollow fiber microtubular membrane has a total surface area of between 5 to 200 cm², preferably a total surface area of between 13 to 92 cm².
d) Maintaining the temperature of the dissolution media using the first temperature controlling unit (12) or a second temperature controlling unit (15) or a combination of both. In yet another embodiment of the present invention a provision for real time monitoring of flow rate of the release media may be provided.
   Optionally circulating the release media through a UV spectrophotometric cell (13) kept between the media reservoir (11) and the hollow fiber module in the direction of the flow of the media for real time detection of an agent in the dosage form.
   Optionally positioning an optical fiber probe in the media reservoir (11), for UV spectrophotometric real time detection of an agent in the dosage form.

In another embodiment of the present invention a system for in-vitro release testing of a drug from dosage form for real time UV monitoring of the drug dispersed from the dosage form may be provided.

The retentate media may have a flow rate of between 2-100 ml/min. Preferably the flow rate is between 4-32 ml/min

The media volume that can be processed by the plurality of hollow fiber microtubular membranes may be between 2 ml to 1000 ml. It is preferable to have the media volume to be processed between 20-100 ml

In another embodiment each hollow fiber module may have a provision for conditioning with appropriate washing fluid. The washing fluid may be selected from washing fluid water, water containing 10% Iso Propyl Alcohol (IPA), 0.5% Sodium Lauryl Sulfate (SLS), tween 80 to enable column reuse. It is preferred to use water containing 10% Iso Propyl Alcohol (IPA) as the washing fluid.

In one embodiment, the module washing/conditioning can be facilitated by circulating the washing media up to 100 ml through microtubules (retentate chamber) with increased pressure between 5-10 psi using throttling valve (19). Permeate chamber shall remain open and the observed permeate flow rate and retentate/permeate back pressure is evaluated by optional pressure sensors/gauges (16/18) to assess the suitability of module for reuse.

In another preferred embodiment, the present invention relates to a microdialysis method for providing an increased permeability area for determination of the diffusible or free concentration of a drug in a dosage form, the method comprising:
a) placing the dosage form in the permeate chamber of a hollow fiber module (17).
b) pumping the dissolution medium from the media reservoir (11) through the retentate chamber (172) of hollow fiber module (17).
c) circulating the permeate media through the plurality of hollow fiber microtubular membranes of the retentate chamber resulting in the release media, wherein release of the drug is occurring in the plurality of hollow fiber microtubular membranes, wherein the plurality of hollow fiber microtubular membrane has a total surface area of between 5 to 200 cm², preferably a total surface area of between 13 to 92 cm².
d) maintaining the temperature of the dissolution media using the first temperature controlling unit (12) or a second temperature controlling unit (15) or a combination of both.
   Optionally circulating the release media through a UV spectrophotometric cell (13) kept between the media reservoir (11) and the hollow fiber module in the direction of the flow of the media for real time detection of an agent in the dosage form.
   Optionally positioning an optical fiber probe in the media reservoir (11), for UV spectrophotometric real time detection of an agent in the dosage form.

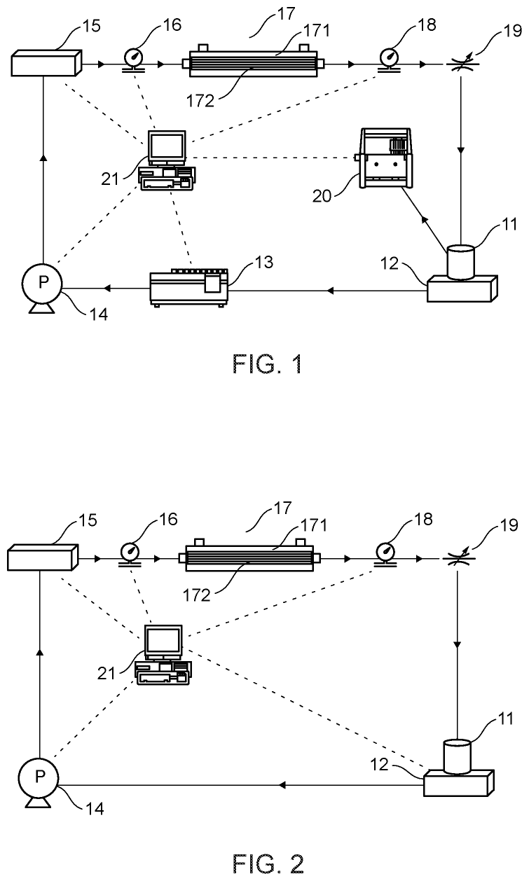

In line with general process flow shown in FIG. 1, in a preferred embodiment, the hollow fiber module can be attached to piston pump and temperature control unit of modified USP-type 4.

In a preferred embodiment, the present invention relates to a microdialysis method for the analysis of the ophthalmic dosage form for complete and faster drug release in a shorter duration of time.

A further embodiment of the present invention relates to a microdialysis method that provides an increase permeability area for faster release of the drug, which enables the complete drug release in a shorter duration of time.

Having described the invention with reference to certain preferred aspects, other aspects will become apparent to one skilled in the art from consideration of the specification. It will be apparent to those skilled in the art that many modifications, both to materials and methods, may be practiced without departing from the scope of the invention.

The detailed experimental parameters suitable for the microdialysis based method are provided by the following examples, which are intended to be illustrative and not limiting of all possible aspects of the invention.

### Example 1—Neafenac Ophthalmic Suspension 0.1% W/V

Based on Dissolution on USP Tpe-4using 500 kD Microdialysis Module)

Brand Name; Nevanac, Manufactured by: Alcon (India Market)

Chromatographic Conditions:

| | |
|---|---|
| Column | Thermo Accucore C18, 50 × 4.6 mm, |
| | 2.6 μm |
| Column temperature | 35° C. |
| Auto injector temperature | 25° C. |
| Wavelength | 238 nm |
| Flow rate | 0.5 mL/Min |
| Injection volume | 20 μL |
| Run time | 10 Min |
| Mobile phase-A | Acetonitrile |
| Mobile phase-B | 10 mM Ammonium formate buffer pH 4.0 |
| Mobile phase-C | Methanol |
| Elution mode | Isocratic |
| Mobile Phase Ratio | Mobile phase A:Mobile Phase B:Mobile |
| | Phase C 27:46:26% V/V |

| | |
|---|---|
| Apparatus | Dissolution USP type-4 (Flow through |
| | cell) Apparatus |
| IVRT Media | 0.75% SLS (Sodium Lauryl Sulphate) |
| | in PBS Buffer pH 7.4 |
| Media Volume | 100 mL |
| Temperature | 35° C. ± 0.5° C. |
| Flow Rate(USP Type-4) | 16 mL/min |
| Microdialysis module details | Size: 500 kD (MOC: mPES) |
| | Surface Area: 20 cm² |
| Sample dilution | 0.5 mL sample to 5 mL with STF |
| | (Simulated tear fluid) buffer pH 7.4 |
| Weight of diluted sample | Approx. 1.2-1.3 |
| loaded (g) | |
| Sampling Volume | 1.0 mL |
| Replenishment Volume | 1.0 mL |
| Time points | 1, 5, 10, 15, 30, 60, 90 & 120 Minutes |

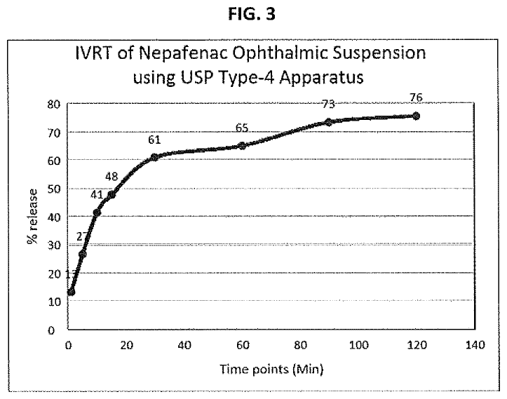

Results: (RLD), N=2 Units are depicted in FIG. 3.

### Example 2—Cyclosporin Ophthalmic Emulsion 0.05%/w/v

(Based on Dissolution UPS Type-4 using 500 kD Microdialysis Module) Brand Name; Restasis, Manufactured by: Allergen (Indian Market)

Chromatographic Conditions:

| | |
|---|---|
| Column | Thermo Accucore C18, 50 × 4.6 mm, |
| | 2.6 μm |
| Column temperature | 80° C. |
| Autoinjector temperature | 25° C. |
| Wavelength | 210 nm |
| Flow rate | 0.5 mL/Min |
| Injection volume | 100 μL |
| Run time | 5 Min |
| Mobile phase-A | 0.1% OPA (Ortho Phosphoric Acid) in |
| | HPLC Water |
| Mobile phase-B | 10% Tert-butyl Methyl Ether in |
| | Acetonitrile |
| Mobile phase-C | Methanol |
| Elution mode | Isocratic |
| Mobile Phase Ratio | Mobile phase A:Mobile Phase B:Mobile |
| | Phase C 32:60:8% V/V |

IVRT Conditions

| | |
|---|---|
| IVRT Media | 0.75% SLS in STF Buffer pH 7.4 |
| Media Volume | 50 mL |
| Temperature | 35° C. ± 0.5° C. |
| Flow Rate (of USP Type-4) | 8 mL/min |
| Microdialysis module details | Size: 500 kD (MOC: mPES) |
| | Surface Area: 20 cm² |
| Sample dilution | 0.6 mL sample to 4.8 mL with |
| | STF buffer pH 7.4 |
| Weight of diluted sample | 1.2-1.3 |
| loaded (g) | |
| Sampling Volume | 1.0 mL |
| Replenishment Volume | 1.0 mL |
| Time points | 5, 10, 15, 30, 45, 60, 90, 120, |
| | 180, 210&300 Minutes |

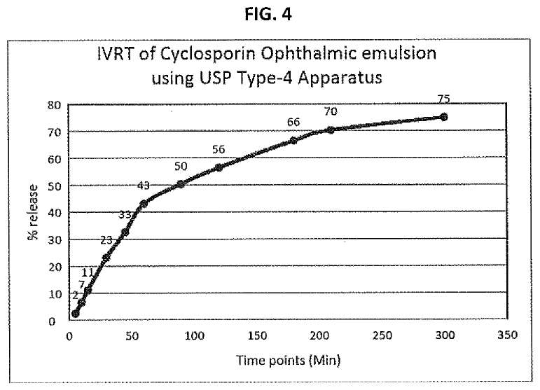

Results: N=3 United are depicted in FIG. 4.

### Example 3.0—Cyclosporin Ophthalmic Emulsion 0.05% w/v

(Based on Dissolutionon UPSTyp4 using 500 kD (Ready to use Spectra/Por, Float-A-Lyzer G2, Spectrum Lab)
Brand Name; Restasis, Manufactured by: Allergen (IndianMarket)
Chromatographic Conditions: Same as example 2
IVRT Conditions:

| | |
|---|---|
| IVRT Media | 0.75% SLS in STF Buffer pH 7.4 |
| Media Volume | 50 mL |
| Temperature | 35° C. ± 0.5° C. |
| Flow Rate (of USP Type-4) | 8 mL/min |
| Float-A-Lyzer details | Size: 500 kD (MOC, Regenerated |
| | cellulose): |
| Sample dilution | 1:5 with STF buffer pH 7.4 in |
| | Float-A-Lyzer |
| Sample Load | 1 ml |
| Sampling Volume | 1.0 mL |
| Replenishment Volume | 1.0 mL |
| Time points | 120, 180, 300, 420, 1200 min |

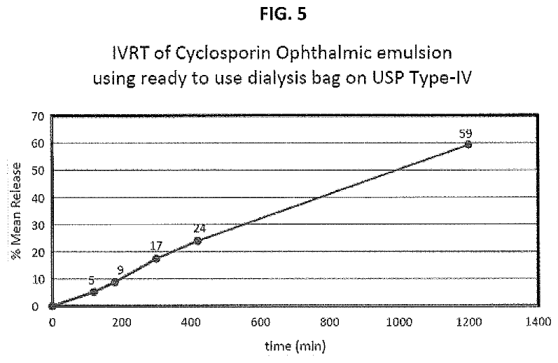

Results: N=3 Units are depicted in FIG. 5.

1. A system for the in-vitro release testing of a drug from a dosage form, the system comprising:
a) a media reservoir (11), consisting of a sampling provision, configured to hold the dissolution media;
b) a first temperature controlling unit (12) kept in contact with the media reservoir (11) for maintaining the temperature of the dissolution media;
c) a pump (14) connected to the media reservoir (11) on one end and a hollow fiber module (17) at the other end, wherein the pump is configured to pump the dissolution media from the media reservoir (11); and
d) a second temperature controlling unit (15) kept in between the media reservoir (11) and the hollow fiber module (17) in the direction of the flow of the media for maintaining the temperature of the dissolution medium.

2. The system of claim 1 wherein the hollow fiber module (17) is comprised of:
a housing;
a permeate chamber (171) for holding the sample;
a retentate chamber (172) comprising a plurality of hollow fiber microtubular membranes, with a total surface area of between 5 to 200 cm².

3. The system according to claim 1, wherein the drug is released from the dosage form into the retentate, wherein the retentate is re-circulated back to the media reservoir (11).

4. A The system according to claim 1, wherein both the temperature controlling units (12) and (15) are used in conjunction.

5-7. (canceled)

8. A The system according to claim 1, wherein the plurality of hollow fiber microtubular membrane, present in the retentate chamber (172) has a total surface area of between 13 to 92 cm².

9. The system according to claim 1, wherein in-vitro release testing of a drug from a dosage form is achieved with automated and/or real-time monitoring of the drug release.

10. The system according to claim 1, wherein in-vitro release testing of a drug from a dosage form is detected with offline monitoring of the drug release.

11-12. (canceled)

13. A The system according to claim 1, wherein the pump configured for circulating the media is selected from peristaltic pump, syringe pump or piston pump.

14. A The system according to claim 2, wherein the plurality of hollow fiber microtubular membrane, present in the retentate chamber (172) comprises of at least five microtubular membranes.

15. The system according to claim 2, wherein the hollow fiber microtubular membrane is comprised of semipermeable membranes selected from Polysulfone, Polyethersulfon, Mixed Cellulose ester or Modified Polyethersulfone.

16. The system according to claim 1, wherein an external fraction collector (20) is connected to the media reservoir (11) for detecting the dispersed dosage form controlled by a firmware.

17. A The system according to claim 1, wherein a UV spectrophotometric cell (13) is connected in between the media reservoir and the hollow fiber module (17) in the direction of the flow of the media for real time monitoring of the release agent.

18. (canceled)

19. The system according to claim 1, wherein a modified United States Pharmacopeia Type 1, 2 or 4 apparatus is used in conjunction with said system.

20. (canceled)

21. A microdialysis method for providing an increased permeability area for determination of the diffusible or free concentration of drug in a dosage form, the microdialysis method comprising:
a) placing the dosage form in the permeate chamber (171) of a hollow fiber module (17) and closing the permeate chamber using end plugs;
b) pumping the dissolution medium from the media reservoir (11) through a retentate chamber (172) of the hollow fiber module (17);
c) circulating the release media through the retentate chamber (172) comprising a plurality of hollow fiber microtubular membranes, wherein release of the drug is occurring in the plurality of hollow fiber microtubular membranes;
d) maintaining the temperature of the dissolution media using a first temperature controlling unit (12) or a second temperature controlling unit (15) or a combination of both;
e) collecting the retentate from the release media through the sampling provision of media reservoir (11); and
f) detecting a component of the dispersed dosage form in the release medium.

22. The microdialysis method according to claim 21, wherein the plurality of hollow fiber microtubular membrane has a total surface area of between 5 to 200 cm².

23. The microdialysis method according to claim 21, wherein the plurality of hollow fiber microtubular membrane has a total surface area of between 13 to 92 cm².

24. (canceled)

25. The microdialysis method according to claim 21, wherein the plurality of hollow fiber microtubular membrane; present in the retentate chamber (172) comprises of at least five microtubules.

26. (canceled)

27. The microdialysis method according to claim 21, wherein the retentate is collected from an external fraction collector (20) connected to the media reservoir (11).

28. The microdialysis method according to claim 21, wherein a component of the dispersed dosage form is detected in the release medium controlled by a firmware (21).

29. The microdialysis method according to claim 21, wherein a component of the dispersed dosage form is detected in the release medium without the usage of the external fraction collector (20) and the firmware (21).

30. The microdialysis method according to claim 21, wherein the release media is circulated through a UV spectrophotometric cell (13) kept between the media reservoir (11) and the hollow fiber module (17) in the direction of the flow of the media for real time detection of the drug in the dosage form.

31. The microdialysis method according to claim 21, wherein an optical fiber probe is positioned in the media reservoir (11), for UV spectrophotometric real time detection of the drug in the dosage form.
